(19) United States
(12) Patent Application Publication
(10) Pub. No.: US 2022/0378330 A1
(43) Pub. Date: Dec. 1, 2022

(54) METHOD AND DEVICE FOR PROVIDING AUDITORY PROGRAM SIMULATING ON-THE-SPOT EXPERIENCE

(71) Applicant: PINE INNOVATION, Hwaseong-si (KR)

(72) Inventors: Dong Wook KIM, Gyeonggi-do (KR); Jae Hee LEE, Seoul (KR); Ju Hee HONG, Gyeonggi-do (KR)

(21) Appl. No.: 17/827,286

(22) Filed: May 27, 2022

(30) Foreign Application Priority Data
Jun. 1, 2021 (KR) 10-2021-0070888

(51) Int. Cl. A61B 5/12; H04S 7/00; G06F 3/16

(52) U.S. Cl. CPC A61B 5/121; H04S 7/303; G06F 3/165

(57) ABSTRACT

A method for providing an auditory program according to an embodiment of the present disclosure may include: decoding, by a processor, first audiovisual data including a target sound to be aurally perceived by a user and an ambient sound reflecting a real-life environment, and playing back the first audiovisual data through a display and a speaker; receiving, by the processor, the user's input based on the result of aural perception of the target sound from the user through a user interface; and changing, by the processor, at least one of parameters of the audiovisual data, based on the user's input, and playing back the audiovisual data through the speaker or the display, or determining a fitting parameter of an assistive listening device, based on the user's input.

### CROSS-REFERENCE TO RELATED APPLICATION(S)

This application is based on and claims priority under 35 U.S.C. 119 to Korean Patent Application No. 10-2021-0070888, filed on Jun. 1, 2021, in the Korean Intellectual Property Office, the disclosure of which is herein incorporated by reference in its entirety.

### BACKGROUND OF THE INVENTION

### 1. Field of the Invention

The present disclosure relates to a device and a method for providing an auditory program simulating on-the-spot experiences and, more specifically, to a device and a method for providing audiovisual data including on-the-spot sounds simulating on-the-spot experiences in daily life as a program for hearing ability testing and rehabilitation.

### 2. Description of the Prior Art

Various kinds of tests exist in connection with the auditory sense. For example, a patient's auditory condition is evaluated while the intensity of pure tones heard by the patient is adjusted with regard to each frequency (pure-tone hearing test), or the auditory threshold and the ability to understand are evaluated with regard to speech sounds (speech hearing test).

Conventional hearing tests have a problem in that the subject's hearing ability is commonly tested and evaluated in an audiometric booth (enclosed soundproof room) of a hospital or the like, and the test does not include on-the-spot sounds occurring in daily life. Therefore, when the subject wears a hearing aid device in daily life after the fitting parameter of the hearing aid device has been adjusted on the basis of the result of evaluation in the soundproof room, the degree of satisfaction may be low.

In addition, each subject may have difficulty in hearing in a different environment (for example, a subject may have difficulty in hearing in an indoor space in which target sounds are heavily reflected, and another subject may have difficulty in hearing if various on-the-spot sounds coexist), but conventional audiometric tests or audiometric rehabilitation schemes fail to consider such differences.

An audiometric rehabilitation device for training the hearing ability according to the prior art (Korean Registered Patent Publication No. 10-2053580) simply adjust the intensity of noise according to the peripheral environment.

However, simple adjustment of the noise intensity is insufficient to rehabilitate a subject to be able to recognize and distinguish between target sounds and on-the-spot sounds occurring in daily life.

### PRIOR ART DOCUMENT

Korean Registered Patent Publication No. 10-2053580 (publicized Dec. 6, 2019)

### SUMMARY OF THE INVENTION

An embodiment of the present disclosure provides a device and a method for testing or rehabilitating a user's hearing ability in an environment similar to daily life.

Another embodiment of the present disclosure provides a device and a method for testing or rehabilitating a user's hearing ability on the basis of audiovisual data including on-the-spot sounds occurring in daily life.

Another embodiment of the present disclosure provides a device and a method for testing or rehabilitating a user's hearing ability on the basis of audiovisual data with which on-the-spot sounds occurring in daily life are synthesized identically or similarly to reality.

Another embodiment of the present disclosure provides a device and a method for generating audiovisual data similar to daily life in which a user actually has difficulty.

Another embodiment of the present disclosure provides a device and a method for testing or rehabilitating a user's hearing ability on the basis of audiovisual data with which a visual element related to a target sound is synthesized.

Aspects of the present disclosure are not limited to the above-mentioned problems, and other aspects and advantages of the present disclosure not mentioned herein will be understood from the following description and will be understood more clearly from embodiments of the present disclosure. in addition, it will be understood that aspects and advantages of the present disclosure can be implemented by means disclosed in the claims and a combination thereof.

A method for providing an auditory program, according to an embodiment of the present disclosure, may include decoding, by a processor, first audiovisual data including a target sound to be aurally perceived by a user and an ambient sound reflecting a real-life environment, and playing back the first audiovisual data through a display and a speaker; receiving, by the processor, the user's input based on the result of aural perception of the target sound from the user through a user interface; and changing, by the processor, at least one of parameters of the audiovisual data, based on the user's input, and playing back the audiovisual data through the speaker or the display, or determining a fitting parameter of an assistive listening device, based on the user's input.

A method for providing an auditory program, according to another embodiment of the present disclosure, may include: receiving, by a processor, a video from a terminal through a network and storing the video in a memory; analyzing the video by the processor; determining, based on the result of analyzing the video, first audiovisual data, which includes a target sound to be aurally perceived by a user and an ambient sound reflecting a real-life environment of the user, in the video by the processor; and transmitting, by the processor, the first audiovisual data to the terminal through a network or transmitting, by the processor, a code capable of playing back second audiovisual data having the same audiovisual parameter as the first audiovisual data to the terminal.

A device for a hearing ability test, according to an embodiment of the present disclosure, may include: a display configured to output a visual result of playing back first audiovisual data; a sound output unit configured to output an aural result of playing back the first audiovisual data; a processor; and a memory electrically connected to the processor and configured to store at least one code executed in the processor, wherein the memory is configured to store codes which, when being executed by the processor, cause the processor to play back the first audiovisual data including a target sound to be aurally perceived by a user and an ambient sound reflecting a real-life environment of the user, and change at least one of parameters for playback of the first audiovisual data, based on an input based on the result of the user's aural perception of the target sound, and play back the first audiovisual data, or determine, based on the input, a fitting parameter of an assistive listening device.

A device for providing an auditory program, according to an embodiment of the present disclosure, may include: a processor; and a memory electrically connected to the processor and configured to store at least one code executed in the processor, wherein the memory is configured to store codes which, when being executed by the processor, cause the processor to determine, based on the result of analyzing an input video, audiovisual data including a target sound to be aurally perceived by a user and an ambient sound reflecting a real-life environment of the user, and transmit, to a terminal, a code capable of playing back the audiovisual data or second audiovisual data having the same audiovisual parameter as the audiovisual data.

A device and a method for testing or rehabilitating hearing ability according to an embodiment of the present disclosure may accurately test and rehabilitate a user's hearing ability while simulating daily life.

A device and a method for testing or rehabilitating hearing ability according to an embodiment of the present disclosure may test and rehabilitate a user's hearing ability while simulating an environment in which the user actually has difficulty, thereby testing and rehabilitating hearing ability applicable to the user's daily life.

Advantageous effects of the present disclosure are not limited to the above-mentioned advantageous effects, and other advantageous effects not mentioned herein will be clearly understood from the following description by those skilled in the art to which the present disclosure pertains.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 illustrates an environment for driving a hearing ability test device 100 and an auditory program providing device 200 according to an embodiment of the present disclosure;

FIG. 2 is block diagram illustrating elements of the hearing ability test device 100 according to an embodiment of the present disclosure;

FIG. 3 is block diagram illustrating elements of the auditory program providing device 200 according to an embodiment of the present disclosure;

FIG. 4 illustrates a method for generating audiovisual data for hearing ability testing or rehabilitation according to an embodiment of the present disclosure;

FIG. 5 and FIG. 6 illustrate an example in which a visual element is synthesized with audiovisual data according to an embodiment of the present disclosure;

FIG. 7 is a flowchart illustrating a method for providing an auditory program simulating an on-the-spot experience according to an embodiment of the present disclosure; and

FIG. 8 is a flowchart illustrating a method for providing an auditory program simulating an on-the-spot experience according to another embodiment of the present disclosure.

### DETAILED DESCRIPTION OF THE EXEMPLARY EMBODIMENTS

Hereinafter, the embodiments disclosed in the present specification will be described in detail with reference to the accompanying drawings, and the same or similar elements are given the same and similar reference numerals, so duplicate descriptions thereof will be omitted. The terms “module” and “unit” used for the elements in the following description are given or interchangeably used in consideration of only the ease of writing the specification, and do not have distinct meanings or roles by themselves. In addition, in relation to describing the embodiments disclosed in the present specification, when the detailed description of the relevant known technology is determined to unnecessarily obscure the gist of the present disclosure, the detailed description may be omitted. Further, the accompanying drawings are provided only for easy understanding of the embodiments disclosed in the present specification, and the technical spirit disclosed herein is not limited to the accompanying drawings, and it should be understood that all changes, equivalents, or substitutes thereof are included in the spirit and scope of the present disclosure.

Terms including an ordinal number such as “first”, “second”, or the like may be used to describe various elements, but the elements are not limited to the terms. The above terms are used only for the purpose of distinguishing one element from another element.

In the case where an element is referred to as being “connected” or “coupled” to any other element, it should be understood that another element may be provided therebetween, as well as that the element may be directly connected or coupled to the other element. In contrast, in the case where an element is “directly connected” or “directly coupled” to any other element, it should be understood that no other element is present therebetween.

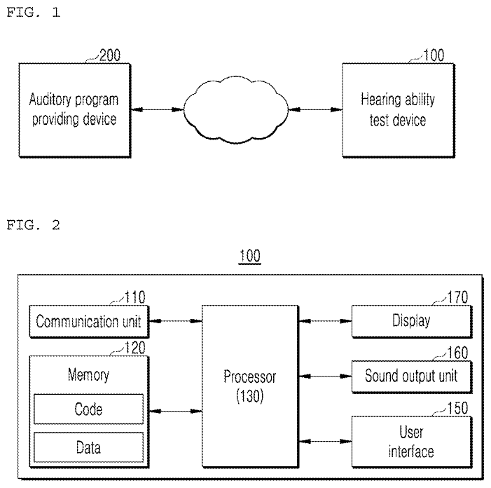

An environment for driving a hearing ability test device 100 and an auditory program providing device 200 according to an embodiment of the present disclosure will be described with reference to FIG. 1.

The hearing ability test device 100 may be implemented as an electronic device of a user, such as a notebook, a smartphone, or a tablet PC, and the type thereof is not specially limited.

In an embodiment, the hearing ability test device 100 may be a terminal based on virtual reality (VR). In this case, the hearing ability test device 100 may play back audiovisual data implemented as a 360-degree video (which includes an image).

The audiovisual data in the present specification includes a video including a visually perceptible element and an aurally perceptible element, and an image including a sound.

The audiovisual data may include an ambient sound or a noise together with a target sound. The target sound may be words suggested to a user in order to test or rehabilitate hearing ability, and may include words or sentences.

A noise in the present specification refers to a sound, such as a white nose or a bubble noise, having a noise level which regularly or irregularly changes in a specific frequency or wide frequency range or is maintained in a predetermined range.

An ambient sound in the present specification may be a sound which is generated in real life and recorded, or a sound generated by imitating the same. In a café environment, the ambient sound may be a sound caused by movement of a chair, a sound caused by grinding coffee beans, conversations between surrounding people, etc.

The hearing ability test device 100 may play back audiovisual data according to a predetermined hearing ability test or rehabilitation program, or may play back the audiovisual data according to a user input.

When the audiovisual data is played back according to a user input, multiple real-life categories 410 may be received through an interface, audiovisual data suitable therefor may be determined or generated, and may be provided to a user with a target sound synthesized therewith.

In an embodiment, the hearing ability test device 100 may receive the audiovisual data from the auditory program providing device 200 and may provide the received audiovisual data to the user.

The auditory program providing device 200 may transfer audiovisual data, appropriately classified into the real-life categories 410 and pre-stored, to the hearing ability test device 100, or may transfer audiovisual data, generated based on a video received from the user, to the hearing ability test device 100.

The hearing ability test device 100 or the auditory program providing device 200 may play back audiovisual data, and may change playback parameters of the audiovisual data, based on a response result (an aural perception result) of the user who has perceived a target sound, and then may play back or synthesize the audiovisual data. For example, a noise level of the audiovisual data may be changed, or a sound parameter reflecting spatial characteristics of a corresponding real-life category may be adjusted. That is, when audiovisual data related to a café is played back, and when the user fails to make a response predetermined times, a target sound or an ambient sound may be synthesized or played back while the level of an echo of an interior space is reduced.

The hearing ability test device 100 or the auditory program providing device 200 may play back the audiovisual data, and may determine a fitting parameter of an assistive listening device, based on a response result (aural perception result) of the user who has perceived a target sound. For example, when the probability of perceiving a target sound in audiovisual data in which an ambient sound M of a specific frequency is synthesized is low, a determination to change a fitting parameter of an assistive listening device related to the corresponding frequency may be made.

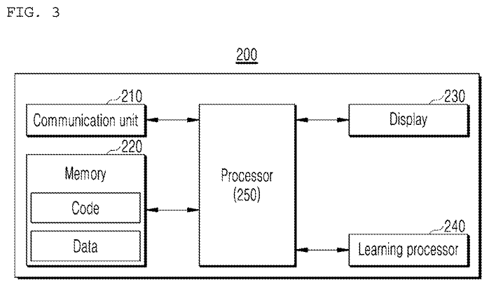

Elements of the hearing ability test device 100 according to an embodiment of the present disclosure will be described with reference to FIG. 2.

The hearing ability test device 100 may include a communication unit 110 configured to communicate with the auditory program providing device 200.

In an embodiment, the communication unit 110 may transmit audiovisual data to a sound output unit 160, implemented as an external device, or a display 170, implemented as an external device.

To this end, the communication unit 110 may include a wireless communication unit or a wired communication unit.

The wireless communication unit may include at least one among a mobile communication module, a wireless Internet module, a short-range communication module, and a position information module.

The mobile communication module may transmit or receive a radio signal to or from at least one among a base station, an external terminal, and a server over a mobile communication network established based on technology standards or communication schemes for mobile communication (for example, Global System for Mobile communication (GSM), Code Division Multi Access (CDMA), Code Division Multi Access 2000 (CDMA2000), Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Long Term Evolution (LTE), Long Term Evolution-Advanced (LTE-A), etc.).

The wireless Internet module refers to a module for wireless internet access, and may be embedded in or disposed outside the hearing ability test device 100. The wireless Internet module may be configured to transmit or receive a radio signal over a communication network based on wireless Internet technologies.

The wireless Internet technologies may include, for example, Wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), Long Term Evolution-Advanced (LTE-A).

The short-range communication module is provided for short-range communication, and may support short-range communication by using at least one among Bluetooth™, Radio Frequency Identification (RFID), Infrared Communication (Infrared Data Association (IrDA)), Ultra-Wideband (UWB), ZigBee, Near Field Communication (NFC), Wireless-Fidelity (Wi-Fi), Wi-Fi Direct, and Wireless Universal Serial Bus (Wireless USB) technologies.

The hearing ability test device 100 may include a memory 120 which stores audiovisual data or which stores code for controlling a processor 130 in order to provide the audiovisual data or generating or synthesize the audiovisual data.

The hearing ability test device 100 may synthesize a target sound with a video based on various real lives, which are provided from the auditory program providing device 200 or stored in advance, to play back audiovisual data.

The hearing ability test device 100 may synthesize, based on a real-life category received from the user, a target sound and an ambient sound, classified as the corresponding category, with an image to play back audiovisual data. The ambient sound and the target sound may be binaural data. In this case, the ambient sound and the target sound may be changed and played back depending on movement of the hearing ability test device 100 or movement of the user's head (which may be based on a VR-based display 170 or an earphone-type sound output unit 160, worn by the user).

In another embodiment, the hearing ability test device 100 may synthesize a target sound with a video provided by the user to play back audiovisual data. For example, in order to make it possible to play back, once or repeatedly, a video recorded in an environment in which the user has trouble in voice perception, the hearing ability test device 100 may synthesize a target sound for the user's perception training with the generated video to play back audiovisual data. In this case, the hearing ability test device 100 may determine, based on a machine learning-based learning model trained to determine, based on an input sound, the type or place of a sound, the type of an ambient sound of a video provided by the user or a place in which the ambient sound is present, and may additionally synthesize the ambient sound with the video, thereby playing back audiovisual data. Furthermore, the level of an ambient sound of a video provided by the user may be determined and adjusted, and then a target sound may be synthesized, or the level of the target sound may be adjusted so as to correspond to the level of the ambient sound and the target sound may be synthesized. Even in an identical video, an ambient sound or a target sound may be adjusted depending on the result of the user's aural perception of the target sound. Therefore, the user's perception ability may be gradually rehabilitated and improved with respect to an identical environment.

The memory 120 may store, as data, an ambient sound classified according to a real-life category, or may sore data related to a sound parameter which reflects a sound characteristic of a corresponding real-life environment according to the corresponding category. When the ambient sound classified according to a real-life category is stored as data, for example, in the case of a café environment, a sound caused by movement of a chair, a sound caused by grinding coffee beans, conversations between surrounding people, etc. may be temporarily or non-temporarily stored as data.

The hearing ability test device 100 may convert a noise or an ambient sound in an image or a video according to a real-life category input from the user so as to be suitable for the corresponding category, thereby playing back audiovisual data. In this case, a sound characteristic according to a spatial characteristic may be reflected based on a sound parameter preconfigured according to the corresponding category, and a noise level may be changed. For example, hearing ability test and rehabilitation may be performed in an environment similar to a real life by reflecting the reflection, absorption, or diffraction of a sound according to spatial characteristics, such as amplifying the reflection and echo of an ambient sound or a target sound in the case of an indoor space, a café, a classroom, or the like, increasing a noise level in the case of the center of a city, or reducing the echo of an ambient sound or a target sound in the case of an outdoor space.

The hearing ability test device 100 may interface a user interface 150 in which the user selects audiovisual data or inputs the result of the user's aural perception of a target sound included in the audiovisual data while testing or rehabilitating a hearing ability by using the audiovisual data.

The user interface 150 may include a microphone for receiving speech from the user, and a user input unit for receiving information which is input from the user.

The user input unit may include a mechanical input means (or a mechanical key, a button, a dome switch, a jog wheel, a jog switch, etc.) and a touch-type input means. In an example, the touch-type input means may include a virtual key, a soft key, or a visual key, which is disposed on the display 170, such as a touch screen, through processing in software, or may include a touch key disposed on a part other than the touch screen.

When the hearing ability test device 100 is implemented as a VR device, the user interface 150 may include a wireless controller capable of indicating and selectively inputting a specific point of audiovisual data displayed on the display 170.

The hearing ability test device 100 may include the sound output unit 160 for outputting an aural element of audiovisual data, and the sound output unit 160 may include a speaker mounted inside the hearing ability test device 100, a wireless earphone implemented as an external device, or a wireless headset. The sound output unit 160 may include a line output terminal for transmitting and outputting sound data to a separate external device.

The hearing ability test device 100 may include the display 170 selectively including a touch interface, and may output an aural element of audiovisual data through the display 170.

The hearing ability test device 100 may include an interface unit for functioning as a passage for a connection to various types of external devices connected to the hearing ability test device 100. The interface unit may include at least one among a wired/wireless data port, a memory card port, a port for connecting a device including an identification module, an audio input/output (I/O) port, a video input/output (I/O) port, and an earphone port.

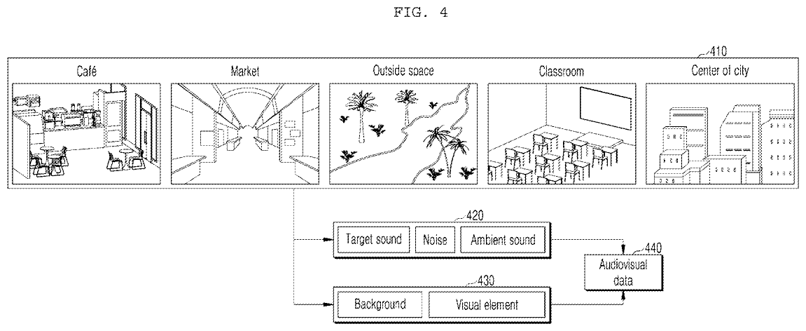

Elements of the auditory program providing device 200 according to an embodiment of the present disclosure will be described with reference to FIG. 3. A detailed description of elements overlapping the above-mentioned elements of the hearing ability test device 100 will be omitted.

The auditory program providing device 200 may include a communication unit 210 for transmitting audiovisual data to the hearing ability test device 100, and a memory 220 for storing input video data or storing codes to be analyzed.

The auditory program providing device 200 may generate audiovisual data based on a video obtained by recording an environment in which the user feels difficulty, and may provide the generated audiovisual data to the hearing ability test device 100. For example, when it is difficult for the user to perceive a voice of a counterpart in a café, the user may input a short video recorded in the corresponding café into the auditory program providing device 200, and the auditory program providing device 200 may analyze the input video to generate audiovisual data simulating an on-the-spot experience similar to the corresponding environment and may provide the audiovisual data to the hearing ability test device 100.

The auditory program providing device 200 may separate sound data from a video which has received from the hearing ability test device 100 or another device, and may transform the sound data into a frequency domain and then may analyze frequency characteristics. For example, a frequency band distribution may be analyzed, or a frequency band change may be analyzed.

The auditory program providing device 200 may determine audiovisual data, based on the result of analyzing the frequency domain of the input video. For example, audiovisual data, which has a frequency band distribution highly similar to that of the video, may be transmitted to the hearing ability test device 100, or audiovisual data, which has a frequency band change similar to that of the video, may be transmitted to the hearing ability test device 100.

In this case, the auditory program providing device 200 may generate audiovisual data by synthesizing noise data having a frequency band distribution or a frequency band change similar to that of the input video with a video classified as a specific real-life category.

The auditory program providing device 200 may input the sound data separated from the input video into a machine learning-based learning model to classify the input video. In this case, the learning model may be learning data in which the sound data is labelled as a real-life category.

Therefore, the auditory program providing device 200 may provide the audiovisual data, classified as a real-life category related to the input video, to the hearing ability test device 100. Furthermore, when there are multiple types of audiovisual data classified as the corresponding real-life category, the auditory program providing device 200 may determine audiovisual data, which has a frequency band distribution or a frequency band change similar to that of the input video, and may provide the determined audiovisual data to the hearing ability test device 100.

In another embodiment, the learning model may be a learning model trained to recognize and classify an object included in an input video.

For example, in the case of a video recorded in a café, people talking with each other at other tables may be recognized and a sound of talking between surrounding people as an ambient sound may be synthesized with audiovisual data, or a coffee machine may be recognized and an ambient sound such as a coffee bean grinding sound, etc. may be synthesized with the audiovisual data. In this case, in the audiovisual data, an ambient sound may be synthesized with a video classified as a specific real-life category, or an ambient sound or visual elements (a coffee machine picture, etc.) classified into recognized objects may be synthesized with a still image classified as a specific real-life category.

The learning model may include CNN, R-CNN (Region based CNN), Convolutional Recursive Neural Network (C-RNN), Long Short-Term Memory (LSTM), Fast R-CNN, Faster R-CNN, region based fully convolutional network (R-FCN), You Only Look Once (YOLO), or a neural network of a Single Shot Multibox Detector (SSD) structure.

The learning model may be implemented as hardware, software, or a combination of hardware and software. When a part or the entirety of a learning model is implemented as software, at least one instruction constituting the learning model may be stored in a memory.

The auditory program providing device 200 may include a separate learning processor 240 for improving training or classification speed of a learning model.

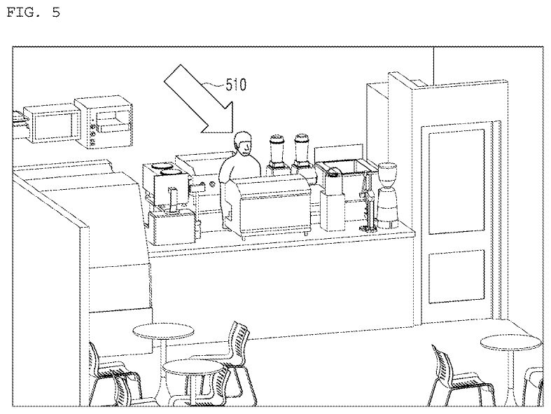

A method for generating or playing back audiovisual data by the hearing ability test device 100 according to an embodiment of the present disclosure will be described with reference to FIG. 4. A detailed description overlapping with the above description will be omitted.

The hearing ability test device 100 may provide an interface in which a user may select real-life categories 410 of audiovisual data to be used to test or rehabilitate a hearing ability.

The hearing ability test device 100 may load, from a storage device, audiovisual data classified into the real-life categories 410 selected by the user, or may request and receive the audiovisual data from the auditory program providing device 200, and then may synthesize a target sound with the audiovisual data and may play back the audiovisual data.

In this case, the audiovisual data may be videos classified into the real-life categories 410 selected by the user and stored, and may be videos obtained by imaging a real-life environment realistically.

The hearing ability test device 100 may reflect sound characteristics according to special characteristics, based on sound parameters preconfigured in real-life categories in which a target sound, a noise, an ambient sound 420 are received from the user, and may change a noise level.

In an embodiment, the audiovisual data may be still images which are classified into the real-life categories 410 selected by the user and stored, and may be still images obtained by photographing real-life environments realistically. In this case, the hearing ability test device 100 may synthesize a preconfigured background image 430 or the like with an input real-life category to generate audiovisual data.

When a display is based on VR, the hearing ability test device 100 may convert a still image or a video into a 360-degree content, and then determine a field of view (FOV) of the user to play back the content so as to be suitable for the FOV.

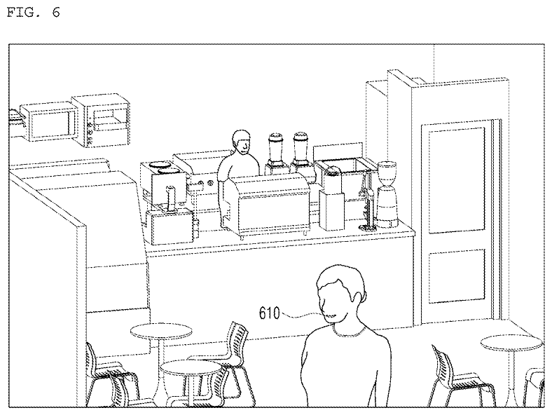

A method for synthesizing or playing back visual element 510 or 610 with audiovisual data according to an embodiment of the present disclosure will be described with reference to FIGS. 5 and 6. A detailed description overlapping with the above description will be omitted.

When synthesizing a target sound with audiovisual data or playing back the target sound, the hearing ability test device 100 or the auditory program providing device 200 may synthesize or play back the target sound such that the target sound has directionality. In this case, the directionality of the target sound may be changed to binaural data, and the binaural data may be synthesized with the audiovisual data or may be played back. Therefore, hearing ability may be tested or rehabilitated in a manner more suitable for real life.

The hearing ability test device 100 or the auditory program providing device 200 may recognize a person in a video or a still image, may change the directionality of the target sound so as to be suitable for the position of the person, and may synthesize the target sound with the audiovisual data or may play back the target sound.

When the target sound is synthesized with the audiovisual data so as to have directionality, an indicator 510 such as an arrow may be synthesized, as a visual element, with the audiovisual data so as to correspond to the directionality.

It may be difficult for a hearing-impaired patient to perceive the directionality of a sound. Particularly, a unilateral hearing-impaired patient may have a greater difficulty. Therefore, in an early test or rehabilitation, a position in which a target sound is generated may be specified and visually presented, thereby aiding the user to be tested or rehabilitated.

The hearing ability test device 100 may not display the indicator 510 when playing back the audiovisual data for the first time, and may play back the audiovisual data such that the indicator 510 is displayed when the result of a response made by the user aurally perceiving a target is wrong. Therefore, the audiovisual data may be variously played back depending on the degree of rehabilitation of the user's hearing ability.

When a realistic avatar generated by an utterer based on an avatar or a generative adversarial network (GAN) with the audiovisual data, the hearing ability test device 100 or the auditory program providing device 200 may synthesize or play back the audiovisual data such that a mouth shape 610 of the avatar or the realistic avatar is changed to correspond to the target sound.

A hearing-impaired patient may more easily perceive the target sound when the mouth shape of the utterer is presented together therewith. Therefore, the audiovisual data may be variously played back depending on the degree of rehabilitation of the user's hearing ability.

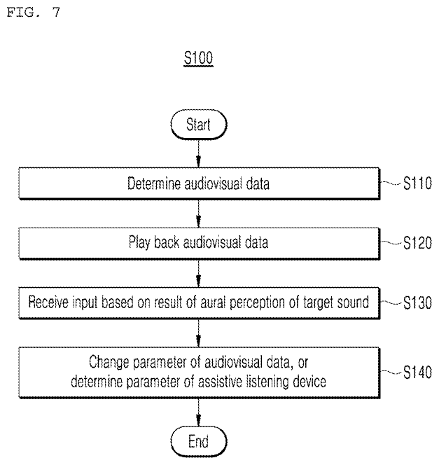

An auditory program providing method according to an embodiment of the present disclosure will be described with reference to FIG. 7. A detailed description overlapping the above description will be omitted.

A hearing ability test device may play back audiovisual data pre-stored or transmitted from an auditory program providing device (S120), wherein the audiovisual data has been determined based on a user input or a predetermined program (S110) and classified into real-life categories.

The hearing ability test device may receive the result of a user's perception of a target sound included in the played-back audiovisual data (S130).

The hearing ability test device may change, based on the perception result, a parameter related to playback of the audiovisual data to play back the audiovisual data, or may determine a fitting parameter of an assistive listening device, based on target sound perception probability according to the perception result (S140). Determining the fitting parameter may be determining an item of a parameter to be changed or determining a numerical value of a specific parameter.

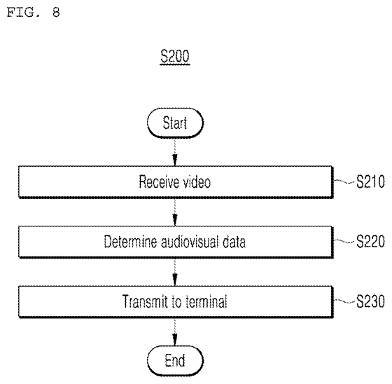

An auditory program providing method according to another embodiment of the present disclosure will be described with reference to FIG. 8. A detailed description overlapping the above description will be omitted.

An auditory program providing device may receive a video from a hearing ability test device or a user terminal (S210). The video may be a video obtained by realistically recording an environment in which a user has actually trouble in perceiving a sound.

The auditory program providing device may determine audiovisual data, based on the result of analyzing a frequency band of the video or the result of classifying the category of the video, based on a learning model (S220). The audiovisual data may be data having frequency characteristics similar to the result of analyzing the frequency band of the video, or may be data pre-stored based on the classification of the category of the video. The auditory program providing device may synthesize ambient sounds or noise data with the audiovisual data, based on the result of analyzing the frequency band of the video or the result of classifying the category of the video, based on a learning model, or may store, based on the result of the analysis or the classification, a parameter for converting ambient sounds or noise data as a parameter data file.

The auditory program providing device may transmit, to the hearing ability test device, the audiovisual data, or the parameter data file for converting the ambient sound or noise data (S230).

The present disclosure as described above may be implemented as codes in a computer-readable medium in which a program is recorded. The computer-readable medium includes all types of recording devices in which data readable by a computer system are stored. Examples of the computer-readable medium include a hard disk drive (HDD), a solid state disk (SSD), a silicon disk drive (SDD), a ROM, a RAM, a CD-ROM, a magnetic tape, a floppy disk, an optical data storage device, and the like. Further, the computer may include a processor of each device.

Meanwhile, the computer program may be specifically designed for the present disclosure, and may be known to and used by those skilled in computer software fields. Examples of the computer program may include machine language code generated by a compiler and high-level language code executable by a computer through an interpreter or the like.

In the specification (particularly, in the claims) of the present disclosure, the term “the” and the indication term similar thereto may correspond to both the singular and the plural. When the present disclosure includes a range, the present disclosure includes an disclosure to which an individual value belonging to the range is applied (unless there is description against it), which means that the detailed description of the present disclosure includes the individual value within the range.

Unless there is clear description of the order of steps included in the method according to the present disclosure or unless indicated otherwise, the steps can be conducted in appropriate order. The present disclosure is not necessarily limited to the order of the steps described therein. All examples or example terms (for example, “etc.”) may be simply used to describe the present disclosure in detail but do not limit the scope of the present disclosure unless the scope of the present disclosure is limited by the claims. Further, those skilled in the art can identify that various modifications, combinations, and changes can be configured according to design conditions and factors within the range of appended claims and equivalents thereof.

Accordingly, the spirit and scope of the present disclosure should not be limited or determined by the above-described embodiments, and it should be noted that not only the claims which will be described below but also their equivalents fall within the spirit and scope of the present disclosure.

What is claimed is:

1. A method for providing an auditory program, the method comprising:
decoding, by a processor, first audiovisual data comprising a target sound to be aurally perceived by a user and an ambient sound reflecting a real-life environment, and playing back the first audiovisual data through a display and a speaker;
receiving, by the processor, the user's input based on a result of aural perception of the target sound from the user through a user interface; and
changing, by the processor, at least one of parameters of the audiovisual data, based on the user's input, and playing back the audiovisual data through the speaker or the display, or determining a fitting parameter of an assistive listening device, based on the user's input.

2. The method of claim 1, further comprising:
receiving, by the processor, real-life environment classification through a network or the user's input before the playing-back of the first audiovisual data; and
synthesizing, by the processor, at least one among the ambient sound, the target sound, and noise data with the first audiovisual data, based on the real-life environment classification.

3. The method of claim 1, wherein the playing-back of the first audiovisual data further comprises:
synthesizing, by the processor, the target sound with a video obtained by imaging a real-life environment realistically; and
decoding, by the processor, the first audiovisual data with which the target sound is synthesized, and playing back the decoded first audiovisual data through the display and the speaker.

4. The method of claim 1, wherein the playing-back of the first audiovisual data further comprises:
receiving, by the processor, second audiovisual data from the user through a network or the user interface;
converting, by the processor, the second audiovisual data received from the user so as to be suitable for a playback device;
synthesizing, by the processor, the target sound with the converted second audiovisual data to generate the first audiovisual data; and
decoding, by the processor, the first audiovisual data with which the target sound is synthesized, and playing back the decoded first audiovisual data through the display and the speaker.

5. The method of claim 4, further comprising:
additionally synthesizing, by the processor, an ambient sound related to a real-life environment of the second audiovisual data with the second audiovisual data, based on a category related to the real-life environment of the second audiovisual data; and
decoding, by the processor, the first audiovisual data with which the ambient sound is synthesized and playing back the decoded first audiovisual data through the display and the speaker.

6. The method of claim 1, wherein the playing-back of the first audiovisual data comprises:
synthesizing, by the processor, a visual element related to the target sound with the first audiovisual data; and
decoding, by the processor, the first audiovisual data with which the visual element is synthesized and playing back the decoded first audiovisual data through the display and the speaker.

7. The method of claim 6, wherein the playing-back of the first audiovisual data comprises:
playing back, by the processor, the target sound such that the target sound has directionality;
synthesizing, by the processor, the visual element related to the directionality with the first audiovisual data; and
decoding, by the processor, the first audiovisual data and playing back the decoded first audiovisual data through the display and the speaker.

8. A method for providing an auditory program, the method comprising:
receiving, by a processor, a video from a terminal through a network and storing the video in a memory;
analyzing the video by the processor;
determining, based on a result of analyzing the video, first audiovisual data, which comprises a target sound to be aurally perceived by a user and an ambient sound reflecting a real-life environment of the user, in the video by the processor; and
transmitting, by the processor, the first audiovisual data to the terminal through a network or transmitting, by the processor, a code capable of playing back second audiovisual data having the same audiovisual parameter as the first audiovisual data to the terminal.

9. The method of claim 8, further comprising:
receiving, by the processor, a result of evaluating an input based on a result of aural perception of the target sound from the terminal; and
changing, by the processor, at least one of multiple parameters for playback of the first audiovisual data, based on the result, or determining, based on the result, a fitting parameter of an assistive listening device.

10. The method of claim 8, wherein the determining of the first audiovisual data comprises determining the first audiovisual data by the processor, based on a result of analyzing a sound of the video in a frequency domain.

11. The method of claim 10, wherein the determining of the first audiovisual data, based on the result of analyzing a sound of the video in a frequency domain, comprises synthesizing, by processor, noise data with the first audiovisual data, based on the result of analyzing a sound of the video in a frequency domain.

12. The method of claim 8, wherein the determining of the first audiovisual data further comprises:
inputting, by the processor, data based on the sound of the video into a machine learning-based learning model to classify the video; and
determining the first audiovisual data by the processor, based on a result of classifying the video.

13. The method of claim 12, wherein the classifying of the video further comprises recognizing, by the processor, an object included in the video, and
the determining of the first audiovisual data, based on the result of classifying the video, further comprises synthesizing, by the processor, the ambient sound and a visual element, preconfigured to relate to the object, with the first audiovisual data.

14. A device for a hearing ability test, the device comprising:
a display configured to output a visual result of playing back first audiovisual data;
a sound output unit configured to output an aural result of playing back the first audiovisual data;
a processor; and
a memory electrically connected to the processor and configured to store at least one code executed in the processor,
wherein the memory is configured to store codes which, when being executed by the processor, cause the processor to
decode the first audiovisual data comprising a target sound to be aurally perceived by a user and an ambient sound reflecting a real-life environment of the user and play back the decoded first audiovisual data through the display and a speaker,
change at least one of parameters for playback of the first audiovisual data, based on the user's input based on a result of the user's aural perception of the target sound, and
play back the first audiovisual data in which the parameter has been changed, or determine, based on the user's input, a fitting parameter of an assistive listening device.

15. The device of claim 14, wherein the memory is configured to further store codes which cause the processor to
convert second audiovisual data received from the user so as to be suitable for a virtual reality (VR) playback environment,
synthesize the target sound with the converted second audiovisual data to generate the first audiovisual data, and
play back the first audiovisual data with which the target sound is synthesized.

16. The device of claim 15, wherein the memory is configured to further store codes which cause the processor to
determine one of ambient sounds classified based on categories of a real-life environment of the second audiovisual data and pre-stored,
further synthesize the ambient sound with the second audiovisual data to generate the first audiovisual data, and
play back the first audiovisual data with which the ambient sound is synthesized.

17. The device of claim 14, wherein the memory is configured to further store codes which cause the processor to
determine a first visual element related to the target sound among multiple visual elements stored in the memory, and
synthesize the first visual element with the first audiovisual data and play back the first audiovisual data

18. The device of claim 17, wherein the memory is configured to further store codes which cause the processor to
play back the target sound such that the target sound has directionality, and
synthesize the first visual element related to the directionality with the first audiovisual data and play back the first audiovisual data.

19. The device of claim 17, wherein the memory is configured to further store a code which causes the processor to synthesize a second visual element related to a lip shape based on the target sound with the first audiovisual data and play back the first audiovisual data.
